(19) United States
(12) Patent Application Publication
(10) Pub. No.: US 2018/0353281 A1
(43) Pub. Date: Dec. 13, 2018

(54) INCREASING BODY FLUID FLOW AT A DESIRED ORIENTATION

(71) Applicant: Invatin Technologies Ltd., Petach Tikva (IL)

(72) Inventor: Udi Nussinovitch, Petach Tikva (IL)

(73) Assignee: Invatin Technologies Ltd., Petach Tikva (IL)

(21) Appl. No.: 16/060,943

(22) PCT Filed: Dec. 26, 2016
(86) PCT No.: PCT/IB2016/058006

§ 371 Date: Jun. 10, 2018

Related U.S. Application Data
(60) Provisional application No. 62/271,419, filed on Dec. 28, 2015.

(51) Int. Cl. A61F 2/07

(52) U.S. Cl. CPC A61F 2/07; A61F 2002/068

(57) ABSTRACT

A device (10) includes an upstream tubular portion (12) from which extends a downstream constriction (14). The downstream constriction (14) is smaller in cross-sectional area than the upstream tubular portion (12). Velocity of fluid flowing through the downstream constriction (14) is greater than velocity of the fluid flowing in the upstream tubular portion (12). A permeable downstream portion (20) is distal to the upstream tubular portion (12). The permeable downstream portion (20) is formed with pores (22) so that the downstream portion (20) is more permeable for fluid flow laterally therethrough than the upstream tubular portion (12).

### FIELD OF THE INVENTION

The present invention relates generally to a methods and devices for amplifying and diverting body fluid flow, and particularly for increasing and diverging body fluid flow at a specific orientation, such as increasing blood flow and its embolic contents to reduce or prevent vascular ischemic events caused due to thrombo-embolism.

### BACKGROUND OF THE INVENTION

Chronic\congestive heart failure (CHF) refers to a state in which the heart is unable to pump enough blood to fulfill the body's need. It may be caused by a variety of reasons, such as cardiac ischemia, valvular insufficiency, cardiotoxic substances, infections, long term effects of hypertension, and others. In developed countries, it is estimated that about 2% of adults have heart failure and in persons over the age of 65, this rate increases to 6-10%. Once developed, heart failure is associated with poor prognosis (with a 5-year survival rate of about 35%). CHF is one of the leading causes of hospitalization, morbidity, decreased quality of life and early mortality worldwide. CHF is known to be associated with an increased risk of stroke (with a 2- to 3-fold increased risk), which is a major cause of disability.

Atrial fibrillation (AFib), which is an irregular electrical and mechanical activity of the atrium, is known to be associated with increased risk for embolic\ischemic stroke. The prevalence of accompanying AFib in CHF patients is about 10% to 17% and may be as high as 50% in patients with advanced stages of heart failure.

Left ventricular (LV) hypokinesia (decreased motion of the myocardium during the cardiac cycle) is a common condition in patients with heart failure, and in patients who sustained a myocardial infarction or other specific cardiac injuries associated with wall motion abnormalities, which may be associated with intra-ventricular thrombus formation. The intra-cardiac thrombi can later be carried along the vascular tree and cause tissue damage following embolization. CHF is associated with increased blood viscosity and overall hyper-coagulability state, further facilitating thrombus formation.

Many valvular conditions, such as rheumatic valve disease, or endocarditis (both infectious and non-infectious) can be associated with a high rate of distal embolization. Also, in the rare event of intra-cardiac tumor (such as in the case of atrial myxoma), systemic embolization occurs frequently (˜50% of cases).

Systemic embolization may also occur due to passage of a venous blood clot to the systemic arterial system via an intra-arterial or intra-ventricular shunting. The presence of a patent-foramen ovale is an example of such common shunting mechanism that may predispose to embolic vascular disease.

Embolic stroke is caused by blockage of an artery by an arterial embolus, i.e., a traveling particle or debris in the arterial bloodstream originating from somewhere else. As noted above, masses may form in the ventricles, atriums, on the cardiac valves, or other places, and detach from these sites, resulting in tissue damage to other organs. Also, emboli may form in deep veins of the legs and be transferred into the systemic circulation (i.e., paradoxical embolism, via an atrial or ventricular septal defect). Of note, stroke was the second most frequent cause of death worldwide in recent years, accounting for more than 6 million deaths. Also, stroke is a leading cause of disability with devastating effects on quality of life.

Several other approaches have been attempted to prevent stroke. Intra-carotid filtering shows great promise but, may decrease blood flow and cannot prevent the consequences of massive emboli (which might plug the vessel completely). Also, these approaches have been attempted as a transient treatment during intravascular interventions, and not as a chronically implanted solution due to the aforementioned reasons.

There are several devices designed to be permanently implanted at the opening of the left atrial appendage (LAA) thus preventing embolism of clots formed in the LAA. In another approach, the LAA can be ligated. Nevertheless, this approach does not prevent embolism of clots formed in the left atrium outside the LAA, or in the left ventricle, as well as emboli that originate from the heart valves or from the right circulatory system.

### SUMMARY OF THE INVENTION

The present invention seeks to increase body fluid flow (such as blood flow or bile flow, for example) and alter specific physical and orientational characteristics of the flow, as is described more in detail below. In some embodiments, the invention amplifies or diverts blood flow to a specific orientation, which may improve CHF symptoms and may prevent vascular ischemic events caused due to thrombo-embolism into specific blood vessels. The invention may be used to prevent stroke in CHF patients, but is not restricted to the treatment of CHF patients alone, and may be used in patients with increased risk to develop thrombo-embolic disease due to other reasons.

The invention is not limited to blood flow and may be used to increase flow of fluids in other body lumens, such as but not limited to, bile, lymph, synovial fluid, urine and others.

In some embodiments, the invention provides a device aimed towards increasing the blood flow at specific axial orientation, thereby improving the cardiovascular activity of the patient, which had been impaired due to low cardiac output, low venous return rate, or other conditions. The device controls flow direction and velocity, and thereby directs the spread of intra-vascular objects, carried within the blood, along the vascular tree at specific orientations away from specific vessels that need to be protected.

In some embodiments, the device includes an intravascular object with a narrow distal end which effectively increases blood flow towards specific orientations, due to the Bernoulli effect. Optionally, the device may be used in conjugation with a blood flow amplification device (e.g., a pump) that could be implanted proximally or distally to the device, externally or intra-cardiac, thereby further improving flow. The smallest lumen of the device does not create a significant increase in flow resistance so as to avoid increasing afterload and exacerbating (or causing) heart failure, or diminish flow overall. The proximal, larger lumen portion of the device may be constructed of a non-penetrable mesh, or partially penetratable mesh or matrix, which ensures that blood content larger than the pre-selected pore size (or the mesh/matrix) will be diverted distally away from vessels that need to be protected from embolism. The device permits a retrograde flow (through the distal penetrable mesh) of small objects only (such as free floating red blood cells, with low kinetic energy due to their lower mass and being less compact compared with larger thrombi). Also, in the case of a partially-penetratable matrix, small objects such as red blood cells will be enabled to flow throughout the proximal portion of the structure. In contrast, thrombi will be accelerated with a specific intra-vascular orientation which is directed away from the vessel that needs to be protected. The relatively high momentum or kinetic energy of these thrombi means that a retrograde flow into the vessel that needs to be protected is less likely to occur.

The device may be used while performing transient procedures associated with increased rate of stroke, such as bypass grafting procedures (or other procedures requiring the use of cardiopulmonary bypass machine), implantation of other intravascular objects (Transcatheter Aortic-Valve Implantation, etc.) and others. The device may be implanted for life, or may be extracted from the vessel if the morbidic condition that necessitated its use has elapsed. The device may be deployed via vascular catheterization or in an open approach.

### BRIEF DESCRIPTION OF THE DRAWINGS

The present invention will be understood and appreciated more fully from the following detailed description taken in conjunction with the drawings in which:

FIG. 1 is a simplified illustration of a device for increasing body fluid flow, in accordance with a non-limiting embodiment of the present invention;

FIG. 2 is a simplified illustration of the device of FIG. 1 implanted in a body lumen, such as the aortic arch, for increasing blood flow at a specific orientation, in accordance with a non-limiting embodiment of the present invention;

FIGS. 3A-3D are simplified illustrations of different downstream endings of the device, in accordance with other embodiments of the invention; and

FIGS. 4A-9B are simplified illustrations of different extracting structures of the device, useful in extracting the device into a catheter for removal from the patient's body, in accordance with different embodiments of the invention, in which the “A” drawing shows the distal portion of the device and the “B” drawing shows extracting the device into the catheter.

### DETAILED DESCRIPTION OF EMBODIMENTS

Reference is now made to FIG. 1, which illustrates a device 10 for increasing body fluid flow, in accordance with a non-limiting embodiment of the present invention.

The invention is described for blood flow in the vasculature, however, the invention is not limited to blood flow and may be used to increase flow of fluids in other body lumens, such as but not limited to, bile, lymph, synovial fluid, urine and others.

Device 10 includes an upstream tubular portion 12 from which extends a downstream constriction 14. Downstream constriction 14 is smaller in cross-sectional area than upstream tubular portion 12. Due to the Venturi effect and Bernoulli's principle, the velocity of a fluid flowing through downstream constriction 14 is increased. In other words, the velocity of fluid flowing through downstream constriction 14 is greater than the velocity of flow in upstream tubular portion 12. The pressure of fluid flowing through downstream constriction 14 is lower than the pressure of fluid flowing in upstream tubular portion 12.

Optionally, a downstream tubular portion 16 may extend distally from downstream constriction 14. In the embodiment of FIG. 1, downstream tubular portion 16 is larger in cross-sectional area than downstream constriction 14.

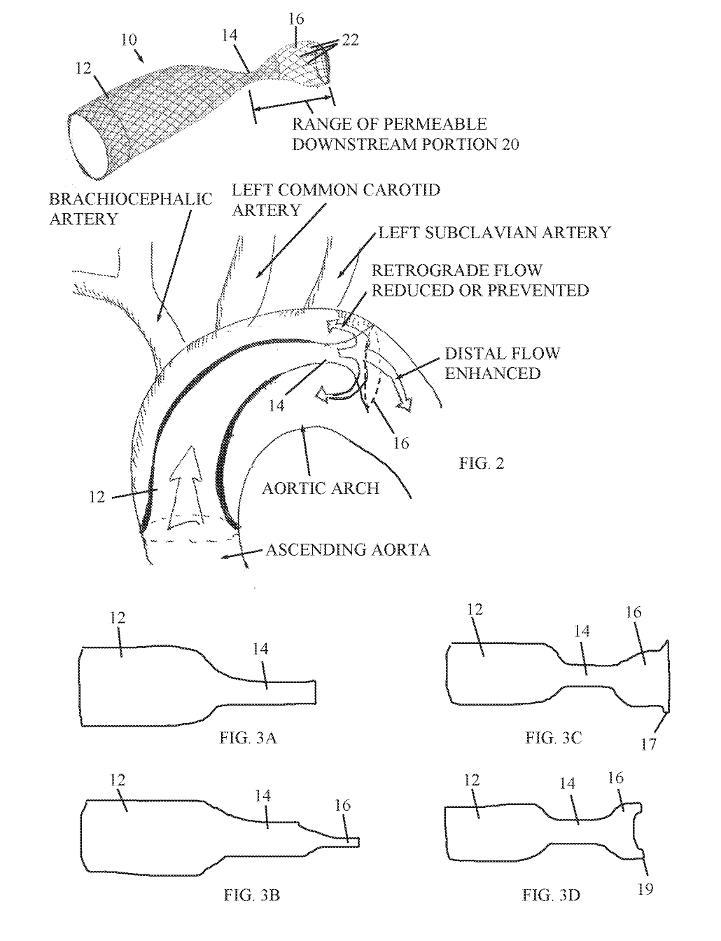

FIGS. 3A-3D illustrate other possible embodiments of the invention. In FIG. 3A, there is no downstream tubular portion after downstream constriction 14. In FIG. 3B, downstream tubular portion 16 is smaller in cross-sectional area than downstream constriction 14, thereby providing further acceleration to the fluid flow. In FIG. 3C, downstream tubular portion 16 has a flared out distal portion 17. In FIG. 3D, downstream tubular portion 16 has a constricted distal portion 19. The flared out distal portion 17 and the constricted distal portion 19 (or other shapes) may provide different flow patterns to the fluid exiting the device, such as maintaining laminar flow, or instead, causing eddy currents or local turbulences to the flow, which may aid in mixing of the constituents of the fluid.

Referring again to FIG. 1, the upstream tubular portion 12 or other parts of device 10 may be self-expanding, balloon-expandable, or constructed of a braided material or wire mesh, metallic or cloth, for example, such as used in self-expanding stents (e.g., made of nitinol or other materials). Alternatively, device 10 may be constructed as an expandable balloon, such as is well known for vascular balloons. The device 10 may be delivered percutaneously as is well known in the art, such as by deployment from a delivery catheter. Device 10 may be anchored in the body lumen, either by the expanded force of the device or by means of fixation anchors.

In accordance with an embodiment of the invention, a permeable downstream portion 20 of device 10 is formed with apertures or pores 22 so that downstream portion 20 is more permeable for fluid flow laterally therethrough than upstream tubular portion 12. In one embodiment, upstream tubular portion 12 is non-permeable. In another embodiment, upstream tubular portion 12 is permeable, but is less permeable than downstream portion 20.

In one embodiment, permeable downstream portion 20 starts from the proximal end of downstream constriction 14 and continues to the distal-most end of device 10. In another embodiment, permeable downstream portion 20 starts after the proximal end of downstream constriction 14, such as an intermediate portion (e.g., halfway, a third of the way, etc.) of constriction 14, and continues to the distal-most end of device 10. In yet another embodiment, permeable downstream portion 20 starts from or after the distal end of downstream constriction 14 and continues to the distal-most end of device 10. In all of the above embodiments, instead of extending to the distal-most end of device 10, the permeable downstream portion 20 may terminate before reaching the distal-most end of device 10. The permeability of permeable downstream portion 20 may be uniformly the same for its entire length, or alternatively, may vary over its length. In a similar manner, the permeability of restricted-penetrability of upstream tubular portion 12 may be uniformly the same for its entire length, or alternatively, may vary over its length.

Each of the above possibilities is a separate embodiment with its own unique flow characteristics to suit a particular flow need.

Reference is now made to FIG. 2, which illustrates device 10 implanted in a body lumen, such as the aortic arch, for increasing and diverting blood flow or any of its contents, in accordance with a non-limiting embodiment of the present invention.

The upstream tubular portion 12 may be located in the ascending aorta and enter the aortic arch. The upstream tubular portion 12 may pass by the inlets to the brachiocephalic artery and left common carotid artery. The downstream constriction 14 may be positioned at or distally past the left subclavian artery. The little or lack of permeability in upstream tubular portion 12 reduces or eliminates flow of emboli into the brachiocephalic artery, the left common carotid artery (and their branches), and to the left vertebral artery. Instead, the emboli are accelerated out of downstream constriction 14 and exit the distal-most end of device 10, with little or no chance of retrograde flow towards the brachiocephalic artery, the left common carotid artery, or the left vertebral artery.

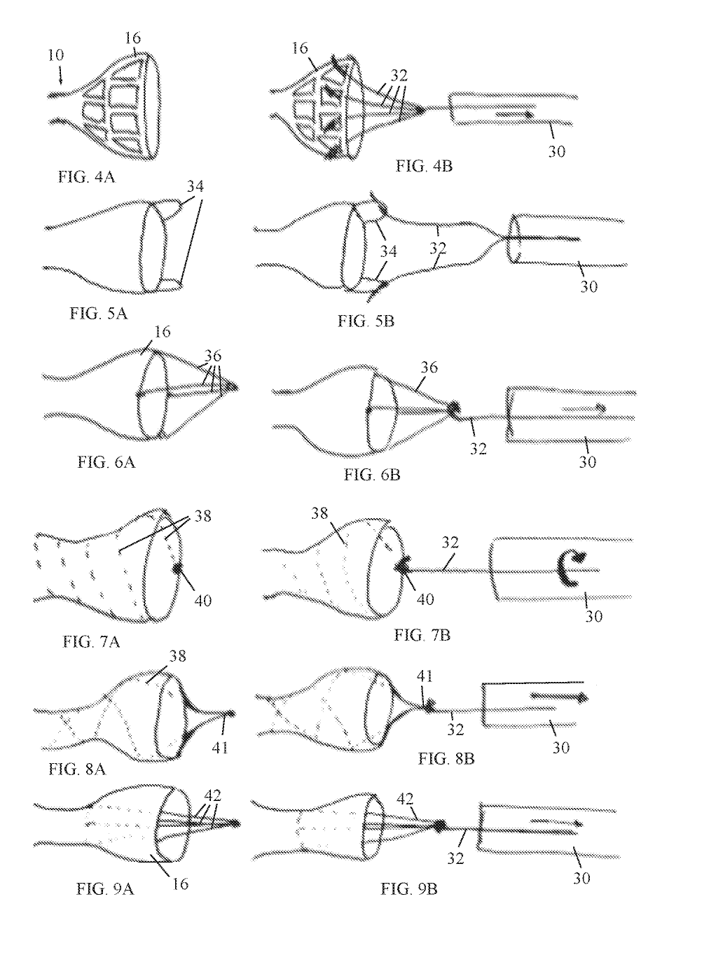

Reference is now made to FIGS. 4A-9B, which illustrate different extracting structures of the device 10, useful in extracting device 10 into a catheter 30 for removal from the patient's body, in accordance with different embodiments of the invention. In all of the embodiments, the extracting structure is located at the downstream tubular portion 16.

In FIGS. 4A-4B, the extracting structure includes one or more catch wires 32 delivered from catheter 30 that grasp pores of the downstream tubular portion 16. By pulling the downstream tubular portion 16 with catch wire or wires 32, the diameter of the downstream tubular portion 16 is reduced and the device 10 can be pulled into catheter 30.

In FIGS. 5A-5B, the extracting structure further includes one or more loops 34 that the catch wires 32 can grasp.

In FIGS. 6A-6B, the extracting structure includes one or more distally facing wires 36 that extend from the periphery of downstream tubular portion 16. The wires 36 can be grasped or hooked by catch wire 32.

In FIGS. 7A-7B, the extracting structure includes one or more helical reinforcements (such as reinforcing wires) 38 embedded in of disposed on the downstream tubular portion 16 plus a receiving member 40, such as a loop or hole, which can be grasped or hooked by catch wire 32. By rotating helical reinforcements 38, the diameter of the downstream tubular portion 16 is reduced and the device 10 can be pulled into catheter 30.

In FIGS. 8A-8B, the extracting structure includes one or more helical reinforcements (such as reinforcing wires) 38, but the receiving member 41 is a protruding wire. The operation is the same as for FIGS. 7A-7B.

In FIGS. 9A-9B, the extracting structure includes one or more distally facing wires 42 that extend from the inner surface of downstream tubular portion 16 (instead of the periphery as in FIGS. 6A-6B). The wires 42 can be grasped or hooked by catch wire 32.

What is claimed is:

1. A device (10) comprising:
an upstream tubular portion (12) from which extends a downstream constriction (14), said downstream constriction (14) being smaller in cross-sectional area than said upstream tubular portion (12), wherein velocity of fluid flowing through said downstream constriction (14) is greater than velocity of the fluid flowing in said upstream tubular portion (12); and
a permeable downstream portion (20) distal to said upstream tubular portion (12), said permeable downstream portion (20) being formed with pores (22) so that said downstream portion (20) is more permeable for fluid flow laterally therethrough than said upstream tubular portion (12).

2. The device (10) according to claim 1, comprising a downstream tubular portion (16) extending distally from said downstream constriction (14).

3. The device (10) according to claim 2, wherein said downstream tubular portion (16) is larger in cross-sectional area than said downstream constriction (14).

4. The device (10) according to claim 2, wherein said downstream tubular portion (16) is smaller in cross-sectional area than said downstream constriction (14).

5. The device (10) according to claim 1, wherein said downstream tubular portion (16) comprises a flared out distal portion (17).

6. The device (10) according to claim 1, wherein said downstream tubular portion (16) comprises a constricted distal portion (19).

7. The device (10) according to claim 1, wherein said upstream tubular portion (12) is self-expanding.

8. The device (10) according to claim 1, wherein said upstream tubular portion (12) comprises an expandable balloon.

9. The device (10) according to claim 1, wherein said upstream tubular portion (12) is non-permeable.

10. The device (10) according to claim 1, wherein said permeable downstream portion (20) starts from a proximal end of said downstream constriction (14) and continues towards a distal-most end of said device (10).

11. The device (10) according to claim 1, wherein said permeable downstream portion (20) starts after a proximal end of said downstream constriction (14) and continues towards a distal-most end of said device (10).

12. The device (10) according to claim 1, wherein said permeable downstream portion (20) starts from or after a distal end of said downstream constriction (14) and continues towards a distal-most end of said device (10).

13. The device (10) according to claim 1, wherein said permeable downstream portion (20) extends up to a distal-most end of said device (10).

14. The device (10) according to claim 1, wherein said permeable downstream portion (20) terminates before reaching a distal-most end of said device (10).

15. The device (10) according to claim 1, wherein permeability of said permeable downstream portion (20) is variable.
